(19) United States
(12) Patent Application Publication
(10) Pub. No.: US 2009/0030241 A1
(43) Pub. Date: Jan. 29, 2009

(54) Novel Amphiphile Acetals

(76) Inventor: Frank-Peter Lang, Hattersheim (DE)

Correspondence Address: CLARIANT CORPORATION;INTELLECTUAL PROPERTY DEPARTMENT, 4000 MONROE ROAD, CHARLOTTE, NC, 28205, US

(21) Appl. No.: 11/886,539

(22) PCT Filed: Mar. 6, 2006
(86) PCT No.: PCT/EP2006/002016

§ 371 Date: Sep. 14, 2007

(30) Foreign Application Priority Data
Mar. 15, 2005 (DE) 10 2005 011 720.1

(51) Int. Cl. C07C 43/04

(52) U.S. Cl. 568/672

(57) ABSTRACT

The invention relates to an amphiphile acetal of formula (1),

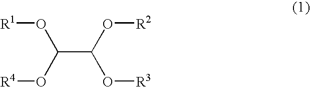

wherein R¹ represents n-C₈-C₂₂-alkyl or iso-C₉-C₁₅-alkyl, R² and R⁴ represent C₁ to C₇-alkyl or have the same meaning as R¹, and R³ represents C₁-C₇-alkyl. The compounds are suitable as surfactants, emulsifiers, demulsifiers, wetting agents or defoaming agents.

Tetramethoxyethane, a bisacetal obtained by reaction of glyoxal with methanol, possesses very interesting solvent characteristics. It is soluble in water in any proportion, like ethanol or isopropanol for example. At the same time, however, it exhibits a significantly more powerful dissolving capacity for fats and oils. For instance, fatty and oily stains are dissolved out of textiles just as well in some instances as with the apolar solvents perchloroethylene or with isoalkanes (white spirit).

This is why tetramethoxyethane appears useful for application in washing and cleaning compositions for textiles (in stain removers or wash boosters for example) or for hard surfaces, for example as a replacement for other organic solvents such as monohydric alcohols (ethanol, isopropanol), di- or more highly hydric alcohols (1,2-propylene glycol, glycerol), ethers, especially glycol ethers, ketones, esters, oligo- and polyalkylene glycols (PEG 300 and PEG 400), terpenes, n- and iso-alkanes and nitrogenous solvents such as N-methylpyrrolidone for example.

Such solvents are used for example in liquid laundry detergents, prewash sprays, stain removers, all-purpose cleaners, glass cleaners or specific cleaners, in the automotive sector for example.

Furthermore, the use of tetramethoxyethane as a substitute for perchloroethylene (also known as PER and perc) or for hydrocarbon solvent (HCS) in dry cleaning is conceivable.

Despite its good dissolving capacity for fats and oils there are specific cases where an even better dissolving capacity for apolar substances and thus a more apolar character for the solvent may be required.

A more apolar character results on exchanging the methyl groups of the tetramethoxyethane for longer alkyl radicals by reacting glyoxal with longer-chain alcohols such as ethanol, propanol or butanol for example to thereby form tetraethoxyethane, tetrapropoxyethane and tetrabutoxyethane. Such symmetrical bisacetals of glyoxal with short-chain linear alcohols having 2 to 6 carbon atoms and with isooctanol are known. One disadvantage with these symmetrical bisacetals, however, is the fact that their solubility in water decreases with increasing carbon chain length.

The present invention accordingly has for its object to provide bisacetals of glyoxal which possess an even better dissolving capacity for fats and oils, or for apolar organic solvents, than known glyoxal acetals without at the same time exhibiting a distinct reduction in their water solubility.

It has now been found that, surprisingly, compounds of the hereinbelow indicated formula (1) have amphiphilic properties which are comparable to those of customary surfactants.

The present invention accordingly provides amphiphilic acetals of the formula (1)

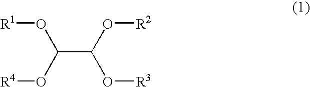

where R¹ is n-C₈-C₂₂-alkyl or iso-C₉-C₂₂-alkyl,
R² and R⁴ are each C₁-C₇-alkyl or have the same meaning as R¹ and R³ is C₁-C₇-alkyl. Preferred meanings for R¹ are n-C₁₀-C₁₈-alkyl, n-C₁₂-C₁₆-alkyl and also iso-C₁₁-C₁₅-alkyl and iso-C₁₃-C₁₅-alkyl. C₁-C₇-Alkyl is preferably methyl, ethyl, n-propyl, isopropyl, n-butyl, isobutyl, tert-butyl.

Of the compounds of the formula 1, the following groups of compounds are preferred: Compounds of the formula 1 wherein R¹ is n-C₈-C₂₂-alkyl, preferably n-C₁₀-C₁₈-alkyl, especially n-C₁₂-C₁₆-alkyl or iso-C₉-C₂₂-alkyl, preferably iso-C₁₁-C₁₅-alkyl, especially iso-C₁₃-C₁₅-alkyl and R², R³ and R⁴ are each C₁-C₇-alkyl.

Compounds of the formula R¹ wherein R¹ and R² are each n-C₈-C₂₂-alkyl, preferably n-C₁₀-C₁₈-alkyl, especially n-C₁₂-C₁₆-alkyl or iso-C₉-C₂₂-alkyl, preferably iso-C₁₁-C₁₅-alkyl, especially iso-C₁₃-C₁₅-alkyl and R³ and R⁴ are each C₁-C₇-alkyl.

Compounds of the formula 1 wherein R¹ and R⁴ are each n-C₈-C₂₂-alkyl, preferably n-C₁₀-C₁₈-alkyl, especially n-C₁₂-C₁₆-alkyl or iso-C₉-C₂₂-alkyl, preferably iso-C₁₁-C₁₅-alkyl, especially iso-C₁₃-C₁₅-alkyl and R² and R³ are each C₁-C₇-alkyl.

The compounds of the formula 1 are preparable in a conventional manner, from glyoxal and the alcohols of the formulae R¹OH, R²OH, R³OH and R⁴OH, by acetalization. The acetalization is preferably carried out in the presence of a small amount of an acidic catalyst.

Useful long-chain alcohols include for example natural fatty alcohols or synthetic alcohols of the type of the oxo process alcohols, Ziegler alcohols or Guerbet alcohols.

Useful acidic catalysts include not only Lewis acids but also Brönstedt acids, examples being zirconium sulfate, sulfuric acid, methylsulfonic acid, p-toluenesulfonic acid, trichloroacetic acid, oxalic acid or acidic ion exchangers.

The glyoxal used is preferably in the form of an aqueous solution, and it is convenient to use the customary technical grade aqueous solutions which have a glyoxal content in the range from 20% to 60% by weight and preferably in the range from 30% to 50% by weight. But it is also possible to use crystalline glyoxal, as a trimer with two moles of water of crystallization, for the acetalization.

Depending on the identity of the alcohol, it can be advantageous to conduct the acetalization in an inert aprotic organic solvent. To shift the reaction equilibrium toward acetalization, the resulting water of reaction is distilled off by an azeotropic distillation.

The compounds of the formula 1 are preparable for example by acetalizing glyoxal with a mixture of a long-chain alcohol and a short-chain alcohol. Depending on whether the bisacetal formed is to contain one, two or three long-chain, lipophilic moieties, the long-chain and short-chain alcohols are used in the molar ratios of 1:3 or 1:1 or 3:1.

The present invention's acetalization of glyoxal can be carried out with an excess of the alcohols R¹OH, R²OH, R³OH and R⁴OH.

Compounds of the formula 1 wherein R¹ and R⁴ are each a long-chain alkyl radical and R² and R³ are each C₁-C₇-alkyl can also be prepared by initially fully acetalizing glyoxal with a single alcohol. The bisacetal thus obtained is then reacted again with glyoxal as described in EP-0 847 976 to obtain the glyoxal monoacetal. This is in turn then reacted again with another alcohol to form the asymmetrical glyoxal bisacetal.

A further way to prepare compounds of the formula 1 consists in transacetalizing a glyoxal bisacetal with long-chain alcohols.

For instance, tetramethoxyethane, the bisacetal of the glyoxal with methanol, may be reacted with 1 to 3 mol of a fatty alcohol in the presence of acidic catalysts, and the methanol released distilled off, to obtain the corresponding mono, di or tri fatty alkyl derivative.

The resulting compounds of the formula 1 exhibit a distinct surfactant behavior, i.e., they exhibit good dissolving capacity for hydrophobic substances, for example for dirt or fats or oils.

Some representative examples of amphiphilic acetals of the formula 1 are:

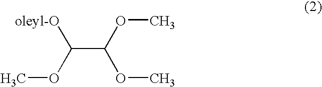

### Formula (2): 1,1,2-trimethoxy-2-oleoxyethane

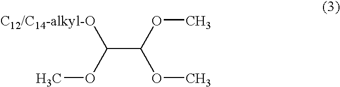

### Formula (3): 1,1,2-trimethoxy-2-C_{12/14}-alkoxyethane

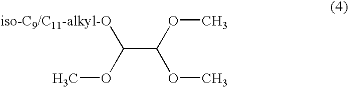

### Formula (4): 1,1,2-trimethoxy-2-C_{9/11}-isoalkoxyethane

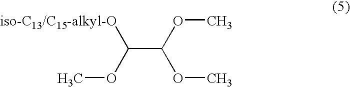

### Formula (5): 1,1,2-trimethoxy-2-C_{13/15}-isoalkoxyethane

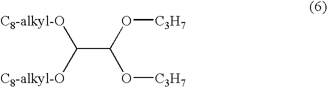

### Formula (6): 1,1-di-C₈-alkoxy-2,2-dipropoxyethane

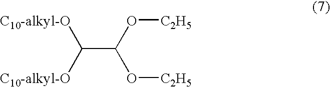

### Formula (7): 1,1-di-C₁₀-alkoxy-2,2-diethoxyethane

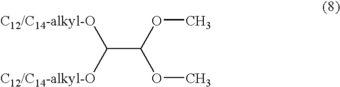

### Formula (8): 1,1-di-C_{12/14}-alkoxy-2,2-dimethoxyethane

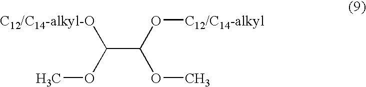

### Formula (9): 1-methoxy-1-C_{12/14}-alkoxy-2-methoxy-2-C_{12/14}-alkoxyethane

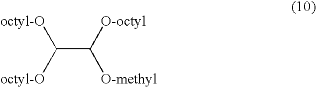

### Formula (10): 1,1,2-tri-C₈-alkoxy-2-methoxyethane

Depending on their method of synthesis, the desired acetals are obtained not only in pure form but as mixtures of various acetals. For instance, 1-C_{12/14}-alkoxy-1,2,2-trimethoxyethane and 1,1,2-tri-C_{12/14}-alkoxy-2-methoxyethane may be by produced in the course of the synthesis of 1,1-di-C_{12/14}-alkoxy-2,2-dimethoxyethane of the formula 8.

The above-described amphiphilic acetals can be used in the following fields: as surfactants, emulsifiers, demulsifiers, dispersants, wetting agents, foamers and defoamers; as washing and cleaning agents in an aqueous liquor for textiles or for hard surfaces composed of coated or uncoated metal, stainless steel, glass, plastics, linoleum, ceramics, porcelain, stoneware, earthenware, wood, concrete, render, or fired clay; in dry cleaning as basic cleaning agent or in spotting agents or cleaning boosters; in the textile industry as leveling agents and dye penetration agents in the dyeing of textiles; as an emulsifier, as wetting agents, as a constituent of size compositions and as lubricants in spinning and weaning, for example; as finishes for textiles to modify their surface constitution, for example with regard to hand, softness, smoothness, hydrophilicity and hydrophobicity; as antistats for synthetic fibers and plastics; as a constituent of lubricants, greases and lubricating oils; as a constituent of metal-processing assistants, for example in drilling, rolling and cutting oils; in crop protectants as emulsifiers, wetting agents and adjuvants for insecticides, fungicides, herbicides; in leather and fur finishing as wetting and washing agents or in admixture with oils as fatliquors; as an additive in papermaking and paper recycling, for example for de-inking; as an emulsifier in emulsion polymerization of, for example, polyvinyl chloride, styrene-butadiene rubber (SBR), acrylonitrile-butadiene-styrene (ABS); as a suspension stabilizer in suspension polymerization; in pigment formulations as auxiliaries, for example in the grinding or dispersing of inorganic and organic color pigments; as additives in colors and coatings, for example as a solubilizer, for superior wetting of the surfaces to be coated, for rheological modification of the formulations, as leveling agents and as flow assistants; in cosmetics, for example as an emulsifier to produce w/o and o/w emulsions; as additives in the flotation to win ores; in building materials such as cement, concrete, gypsum, for example as superplasticizers or as air pore formers; to produce bitumen emulsions, for example as adhesion promoters; as additives in the primary and secondary production of petroleum, for example as emulsion breakers/demulsifiers to separate water from crude oil or as an addition to flooding water to enhance the crude oil yield; and as power fuel additives.

1. An amphiphilic acetal compound of formula (1) 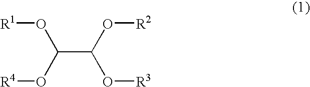
where R¹ is n-C₈-C₂₂-alkyl or iso-C₉-C₂₂-alkyl,
R² and R⁴ are each C₁-C₇-alkyl or have the same meaning as R¹ and R³ is C₁-C₇-alkyl.

2. The compound of claim 1 wherein R¹ is n-C₁₀-C₁₈-alkyl or iso-C₁₁-C₁₅-alkyl, R² and R⁴ are each C₁-C₇-alkyl or have the same meaning as R¹ and R³ is C₁-C₇-alkyl.

3. The compound of claim 1 wherein R¹ is n-C₁₂-C₁₆-alkyl or iso-C₁₃-C₁₅-alkyl, R² and R⁴ are each C₁-C₇-alkyl or have the same meaning as R¹ and R³ is C₁-C₇-alkyl.

4. The compound of claim 1 wherein R¹ is n-C₈-C₂₂-alkyl or iso-C₉-C₂₂-alkyl, and R², R³ and R⁴ are each C₁-C₇-alkyl.

5. The compound of claim 1 wherein R¹ is n-C₁₀-C₁₈-alkyl or iso-C₁₁-C₁₅-alkyl, and R², R³ and R⁴ are each C₁-C₇-alkyl.

6. The compound of claim 1 wherein R¹ is n-C₁₂-C₁₆-alkyl or iso-C₁₃-C₁₅-alkyl, and R², R³ and R⁴ are each C₁-C₇-alkyl.

7. The compound of claim 1 wherein R¹ and R² are each n-C₈-C₂₂-alkyl or iso-C₁-C₂₂-alkyl and R³ and R⁴ are each C₁-C₇-alkyl.

8. The compound of claim 1 wherein R¹ and R² are each n-C₁₀-C₁₈-alkyl or iso-C₁₁-C₁₅-alkyl and R³ and R⁴ are each C₁-C₇-alkyl.

9. The compound of claim 1 wherein R¹ and R² are each n-C₁₂-C₁₆-alkyl or iso-C₁₃-C₁₅-alkyl and R³ and R⁴ are each C₁-C₇-alkyl.

10. The compound of claim 1 wherein R¹ and R⁴ are each n-C₈-C₂₂-alkyl or iso-C₁-C₂₂-alkyl and R² and R³ are each C₁-C₇-alkyl.

11. The compound of claim 1 wherein R¹ and R⁴ are each n-C₁₀-C₁₈-alkyl or iso-C₁₁-C₁₅-alkyl and R² and R³ are each C₁-C₇-alkyl.

12. The compound of claim 1 wherein R¹ and R⁴ are each n-C₁₂-C₁₆-alkyl or iso-C₁₃-C₁₅-alkyl and R² and R³ are each C₁-C₇-alkyl.

13. A method for preparing an agent composition selected from the group consisting of a surfactant, an emulsifier, a demulsifier, a dispersant, a wetting agent, a foamer, and a defoamer, said method comprising adding to the agent composition the compound of claim 1.

14. A method for cleaning a hard surface or a textile, said method comprising contacting said hard surface with a cleaning agent in an aqueous liquor comprising the compound of claim 1, wherein the hard surface is selected from the group consisting of composed of coated or uncoated metal, stainless steel, glass, plastics, linoleum, ceramics, porcelain, stoneware, earthenware, wood, concrete, render and fired clay.

15-34. (canceled)
